(19) United States
(12) Patent Application Publication
(10) Pub. No.: US 2007/0006886 A1
(43) Pub. Date: Jan. 11, 2007

(54) Mouth closure

(75) Inventors: David A. Bates, Libertyville, IL (US); Joanne N. Crawford, Wilmette, IL (US)

Correspondence Address: DAVID BATES, 911 E. ROCKLAND RD., LIBERTYVILLE, IL, 60048, US

(73) Assignee: Daybreak Products, LLC, Libertyville, IL (US)

(21) Appl. No.: 11/159,636

(22) Filed: Jun. 24, 2005

(51) Int. Cl. A61F 11/00

(52) U.S. Cl. 128/857

(57) ABSTRACT

A system for holding the mouth of a human closed when lying in the supine position. Such a system can be used for deceased persons with the tendency for the mouth to relax open. Use of this system adds dignity to the cadaver for viewing by others prior to the embalming process.

### REFERENCES CITED

U.S. PATENT DOCUMENTS

| | | | |
|---|---|---|---|
| 1519915 | December 1924 | Johnson | 602/902 |
| 1587558 | June 1926 | Sheffield | 602/902 |
| 2416411 | February 1947 | Sharbaugh et al. | 128/206 |
| 3513482 | May 1970 | Holden | 2/421 |
| 3776244 | December 1973 | Morgan | 2/171 |
| 4051556 | October 1977 | Davenport et al. | 2/421 |
| 4651356 | March 1987 | Zide | 2/421 |
| 4692947 | September 1987 | Black et al. | 2/425 |
| 4766610 | August 1988 | Mattes | 2/909 |

### BACKGROUND OF THE INVENTION

1. Field of the Invention

This invention relates to tendency of the human mouth to relax in the open position when a body is laying flat on its back in the supine position with the head pointed upward. This condition of an open mouth is particularly common for a recently deceased person prior to the embalming process.

For a recently deceased person the typical practice is to use wrapped towels or a crude strap around the head to hold the mouth shut. Present devices use chin and head straps are complicated, cumbersome and aesthetically un-appealing. The intent of this invention is to add dignity to the cadaver for display to loved ones via an un-obtrusive or transparent holding device.

2. Description of the Prior Art

Previous devices relate to chin straps and are specifically used for corrective and protective measures. Such chin straps use complex straps and fasteners. For example, U.S. Pat. No. 1,519,915, issued Dec. 16, 1924 to Walter G. Johnson, discloses a corrective mouth breather having a chin strap with a chin cup in combination with a mouth closure and a plurality of straps for forcing a user to breathe only through the nose.

Similar to that of the foregoing corrective mouth breather is the jaw bracing and setting appliance shown in U.S. Pat. No. 1,587,558, issued Jun. 8,1926 to Elise H. Sheffield. Sheffield discloses an appliance having a chin strap with a chin cup. The chin cup has an oval shaped aperture in which the apex of the chin is placed and two straps for securing the device to the user.

A retention device disclosed in U.S. Pat. No. 3,776,244, issued Dec. 4, 1973 to Willis E. Morgan has a non-slip retention means using a chin strap secured by a plurality of straps via hook and loop fasteners. U.S. Pat. No. 3,572,329, issued on Mar. 23,1971 to Irvin S. De Woskin discloses a chin strap with two straps for securing it to a protective headgear.

U.S. Pat. No. 4,651,356, issued Mar. 24, 1987 to Robert M. Zide, discloses a helmet chin strap with a chin cup secured via four straps.

U.S. Pat. No. 5,123,121, issued Jun. 23,1992 to Lester V. Broersma discloses a helmet retention system with straps that are adjustably engaged via glide rings.

Another patent of interest is U.S. Pat. No. 5,183,059, issued Feb. 2,1993 to David Leonardi discloses an eye shield retention system with straps looped through eyelets and secured via hook and loop fasteners.

U.S. Pat. No. 5,361,416, issued Nov. 8,1994 to Steven C. Petrie et al. discloses a headcover and chin straps which engage a set of D-rings. Said device is used for treating sleep apnea.

None of the above inventions and patents, taken either singly or in combination, is seen to describe the instant invention as claimed.

### SUMMARY OF THE INVENTION

The present invention relates to a system for holding a human mouth closed when the human body is laying flat on its back in the supine position. The system comprises a rigid or semi-rigid part that wedges between the underside of the lower jawbone and the upper chest.

The part is held in place by straps that extend around behind the neck. The straps may be connected behind the neck with any common method. One possibility of connecting the straps is by use of hook and loop fasteners.

The present invention uses un-obtrusive and/or transparent parts with smooth corners and edges.

### BRIEF DESCRIPTION OF THE DRAWINGS

The invention is described in greater detail in the following description of examples embodying the best mode of the invention taken in conjunction with the drawing figures, in which:

FIG. 1 is perspective view showing the rigid or semi-rigid part with straps attached.

FIG. 2 is a side view showing the preferred form of the invention with the mouth closure part in place on the head of a human laying in the supine position with head upward with the straps holding the mouth closure in place.

FIGS. 3-5 show alternate forms of the mouth closure part.

### DESCRIPTION OF EXAMPLES EMBODYING THE BEST MODE OF THE INVENTION

FIG. 1 illustrates the first form of the invention with the mouth closure part 10, straps 12 and fasteners 24. Part 10 has a large opening 22 in the central area of part 10 to accommodate the thyroid cartilage (Adam's apple) common in the human male. Part 10 has rounded edges 26 that extend around the entire periphery of part 10 to minimize deformation of the flesh.

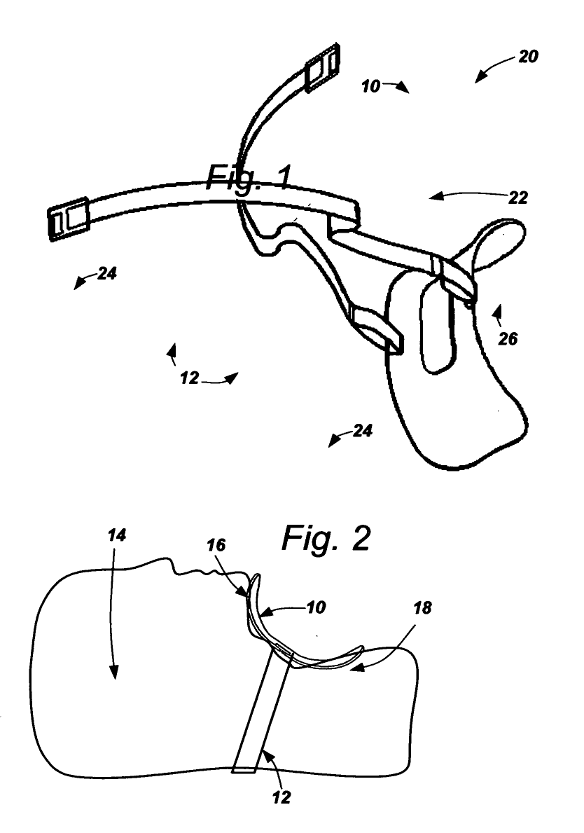

FIG. 2 shows the position of the invention on the head 14. The combination of part 10 with straps 24 can be assembled onto the head in either direction such that surface 20 (FIG. 1), which is the same on both distal ends of part 10, can rest either on the underside of the jawbone (chin) 16 as shown in FIG. 2 or on the upper chest along the upper sternum (breastbone) between the clavicles (collarbones) 18. This preferred form of the invention is considered a non-polar assembly since it can be positioned on the head in either direction since part 10 is symmetrical in both vertical and horizontal planes.

FIG. 3 shows an alternate form of part 10 of the invention. This part is polar since it would be positioned on the head in one direction only with the smaller upper end against the underside of the jawbone (chin) 16 and the larger lower end resting on the upper chest along the upper sternum between the clavicles 18. The alternate part 10 shown in FIG. 3 has large openings. This part may be a wireform.

FIG. 4 shows another alternate form of part 10 which again is polar with the upper end against the underside of the jawbone (chin) 16 and the two lower legs resting on the upper chest along the upper sternum between the clavicles 18.

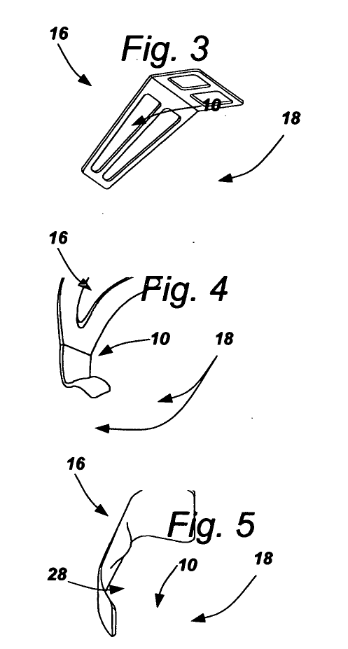

FIG. 5 illustrates another alternate form of part 10 which again is upper end against the underside of the jawbone (chin) 16 and the lower larger end resting on the upper chest along the upper sternum between the clavicles 18. This part shows a bulge 28 in the central area of part 10 again to accommodate the thyroid cartilage.

All illustrations of part 10 can be made of metal. Fabrication from plastic allows clear transparency. Fabrication from metal wire(form) can also provide some minimal visual obstruction.

Various changes can be made to the invention without departing from the spirit thereof or scope of the following claims.

What is claimed is:

1. A system for holding the mouth of a human closed when in the supine position comprising:
a. a rigid or semi-rigid part to impinge on the underside of the lower jaw bone,
b. said part bending and extending down along the neck to the upper chest, and
c. said part bending again to lay along the upper sternum (breastbone), between the clavicles (collarbones).

2. A system according to claim 1 in which the part is made from either plastic or metal.

3. A system according to claim 1 in which the part has comers, radii and smooth edges.

4. A system according to claim 1 in which the part is clear, transparent when made from plastic.

5. A system according to claim 1 in which the part has large openings when made from metal.

6. A system according to claim 1 in which said part has a central large opening.

7. A system according to claim 1 in which said part has side openings for attachment of holding straps.

8. A system according to claim 1 in which said part has a strap or straps attached at the sides.

9. A system according to claim 8 in which straps are flexible, clear, transparent.

10. A system according to claim 8 in which straps have an attachment mechanism to attach to each other at the distal ends of the straps from part in claim 1; attachment at the back of the neck.
